(19) United States
(12) Patent Application Publication
(10) Pub. No.: US 2023/0234843 A1
(43) Pub. Date: Jul. 27, 2023

(54) SYSTEMS AND METHODS FOR PRODUCING CARBON-NEGATIVE GREEN HYDROGEN AND RENEWABLE NATURAL GAS FROM BIOMASS WASTE

(71) Applicant: Yosemite Clean Energy, LLC, Mariposa, CA (US)

(72) Inventors: Thomas Garrett Hobby, Fresno, CA (US); Robert David Jackson, Kingsburg, CA (US)

(21) Appl. No.: 18/095,939

(22) Filed: Jan. 11, 2023

Related U.S. Application Data
(60) Provisional application No. 63/303,629, filed on Jan. 27, 2022.

(51) Int. Cl. C01B 3/16; C01B 3/56; C01B 3/50; C01B 32/50; C10J 3/82; C10L 3/10; C07C 5/03

(52) U.S. Cl. CPC C01B 3/16; C01B 3/56; C01B 3/506; C01B 32/50; C10J 3/82; C10L 3/103; C07C 5/03; C01B 2203/0283; C01B 2203/042; C01B 2203/046; C01B 2203/048; C01B 2203/0475; C01B 2203/062; C10J 2300/1612; C10J 2300/1618; C10J 2300/0916; C10J 2300/1662; C10L 2290/542; C10L 2200/0469

(57) ABSTRACT

Methods and systems for producing carbon-negative hydrogen and renewable natural gas from biomass are included herein. In an embodiment, the method may include gasifying biomass in a gasification unit to form a first stream comprising syngas. The syngas may include methane, hydrogen, carbon dioxide, carbon monoxide, ethylene, and water. The method may also include reacting the carbon monoxide with water in the presence of a catalyst to form a second stream. The second stream may include a greater hydrogen concentration than the first stream. The method may further include separating at least a portion of the second stream to form a hydrogen stream and a natural gas stream. The hydrogen stream may have a greater concentration of hydrogen than the second stream. The natural gas stream may have a greater concentration of methane than the second stream.

### CROSS-REFERENCE TO RELATED APPLICATIONS

This application claims the benefit of U.S. Provisional No. 63/303,629 filed on Jan. 27, 2022, entitled “SYSTEMS AND METHODS FOR PRODUCING CARBON-NEGATIVE GREEN HYDROGEN AND RENEWABLE NATURAL GAS FROM BIOMASS WASTE,” the entire contents of which are incorporated herein by reference.

### FIELD

The present application generally relates to methods and systems for producing carbon-negative green hydrogen and renewable natural gas from biomass waste.

### BACKGROUND

Climate change remains one of the biggest challenges for humankind. In order to slowdown climate change and reverse the negative effects of climate change greenhouse gas emissions must be reduced drastically and by some accounts, go to a negative carbon emission levels in the near term to make any meaningful impact. Decarbonization of the power sector is already underway with the tools currently available. There has been significant progress in decarbonizing electricity in recent years, exemplified by a 33 percent (800 million ton) decline in U.S. electricity system emissions by 2019 from their peak in 2007 despite a nearly identical generation level. The electric power sector is often regarded as the “easiest” sector to decarbonize, compared with highly diffuse sectors such as transportation, because of the large number of solutions available and the relative ease of transitioning a relatively limited number of generally centralized assets.

The transportation sector in the United States is the largest direct source of greenhouse gases (industry ranks higher when counting both its direct and indirect emissions). In California about 40 percent of the greenhouse gas (GHG) emissions is from the transportation sector. Most transportation emissions are carbon dioxide (CO₂) produced by the combustion of fossil fuels. Methane and nitrous oxide are also emitted as by-products of combustion. Total transportation sector emissions rose 29 percent from 1990 to 2005, driven largely by vehicle miles traveled (VMT) increases in road transport.

Countries around the world, including the U.S. federal government are taking initiatives to reduce GHG emissions from the transportation sector. These initiatives and policies fall in the following major categories: (1) reducing emissions from light duty vehicles, (2) reducing emissions from heavy duty vehicles, (3) increasing the use of lower carbon fuels, and (4) Reducing the number of vehicle miles traveled.

Modern vehicles are more energy efficient compared to similar vehicles even from a couple of decades ago. But that is not adequate because there are lot more vehicles on the road now. The world now needs zero emission vehicles (ZEV) and negative carbon emission fuels. In order to stabilize global warming and climate change at any level, emissions of carbon dioxide, the main greenhouse gas, need to be eliminated; reducing them is not enough.

This will require broad adoption and deployment of ZEV technologies and use of: (1) carbon-negative green hydrogen, and (2) renewable natural gas as transportation fuels.

### SUMMARY

Various examples are described for systems and methods for producing carbon-negative hydrogen and renewable natural gas from biomass. The method for producing carbon-negative hydrogen and renewable natural gas from biomass may include gasifying biomass in a gasification unit to form a first stream comprising syngas, which is sometimes referred to as producer gas. The syngas may include methane, hydrogen, carbon dioxide, carbon monoxide, ethylene, and water. The method may also include reacting the carbon monoxide with water in the presence of a catalyst to form a second stream. The second stream comprises a greater hydrogen concentration than the first stream. In some embodiments, the reacting step may also include hydrogenating the ethylene in the presence of the catalyst to form ethane. The second stream may include a greater ethane concentration than the first stream.

The method may also include separating at least a portion of the second stream to form a hydrogen stream and a natural gas stream. The hydrogen stream may have a greater concentration of hydrogen than the second stream, and the natural gas stream may have a greater concentration of methane than the second stream. For example, the natural gas stream may have a methane concentration of 70-80% by volume. The natural gas stream may be a pipeline-quality gas that is interchangeable with conventional natural gas.

In some embodiments, the separating step may include separating at least a portion of the second stream into the hydrogen stream and a tail gas stream and separating the natural gas stream from the tail gas stream. For example, the separating step may include a pressure swing adsorption process and/or a cryogenic separation step. In some embodiments, steam may be added to the first stream prior to the reacting step. The method may further include capturing waste carbon dioxide and sequestering the waste carbon dioxide such that the production of the carbon-negative hydrogen and the renewable natural gas from biomass is a net-negative carbon emission process for both fuel types with corresponding carbon intensity emissions indices. Optically, the method may include removing at least one of tar or ammonia from the first stream prior to the reacting step.

A system for producing carbon-negative hydrogen and renewable natural gas from biomass is also provided. The system may include a gasification unit, a syngas reaction unit, a hydrogen extraction unit, and a natural gas separation unit. The gasification unit may gasify biomass to form a first stream comprising syngas and a flue gas stream, wherein the syngas includes methane, hydrogen, carbon dioxide, carbon monoxide, ethylene, and water. The gasification unit may include a gasifier and a combustion chamber. The gasifier may be fluidized by superheated steam generated by combusting a portion of the flue gas stream in the combustion chamber. In some embodiments, the system may further include a flue gas processing unit. The flue gas processing unit may receive flue gas from the gasification unit and clean the flue gas.

The syngas reaction unit may react the carbon monoxide with water in the presence of a catalyst to form a second stream. For example, the syngas reaction unit may include a CO-shift reactor. The second stream may have a greater hydrogen concentration than the first stream. Optionally, the reaction unit may also hydrogenate the ethylene in the presence of the catalyst to form ethane, wherein the second stream may have a greater ethane concentration than the first stream. In some embodiments, prior to introducing the first stream to the syngas reaction unit, a steam stream may be added to the first stream.

The hydrogen extraction unit may separate at least a portion of the second stream to form a hydrogen stream and a tail gas stream. For example, the hydrogen extraction unit may include a pressure swing adsorption unit. The hydrogen stream may have a greater concentration of hydrogen than the second stream. In some embodiments, prior to the hydrogen extraction unit, the system may include a carbon-dioxide separation unit. The carbon-dioxide separation unit may include at least a carbon-dioxide scrubber and may remove at least a portion of carbon dioxide from the second stream.

The natural gas separation unit separates the tail gas stream to form a natural gas stream. For example, the natural gas separation unit may include a cryogenic separation unit. wherein the natural gas stream comprises a greater concentration of methane than the second stream. The system may also include a syngas cleaning unit including at least one scrubber. The scrubber(s) may remove from the second stream at least one of tar or ammonia. In some embodiments, the system may further include a carbon capture and sequestration unit. The carbon capture and sequestration unit may capture carbon dioxide produced by the system and remove the carbon dioxide from the system to result in the system having a net negative carbon emission.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates an example plant process according to an embodiment of the present invention.

### DETAILED DESCRIPTION

Examples are described herein in the context of systems and methods for producing carbon-negative green hydrogen and renewable natural gas from biomass waste. Those of ordinary skill in the art will realize that the following description is illustrative only and is not intended to be in any way limiting. Reference will now be made in detail to implementations of examples as illustrated in the accompanying drawing. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

The conventional method of generating green hydrogen has been to use an electrical current to separate the hydrogen from the oxygen in water. By definition, ‘green hydrogen’ is generated from electricity produced by a primary renewable source such as wind or solar. But both of these renewable sources of electricity have intermittency issues. Moreover, to generate green hydrogen in large quantities to support the transportation sector it will require massive amounts of renewable power and massive amounts of electrical energy storage capacity. Due to these current constraints, green hydrogen currently accounts for less than 1 percent of total annual hydrogen production. The process of converting wood biomass waste to green hydrogen described here is an innovative process and provides a new alternative method for producing carbon negative biofuels. The systems and methods described herein provide for a more efficient and effective way of converting inputs (Biomass) to biofuels over conventional techniques.

Renewable natural gas (RNG) is a pipeline-quality gas that is fully interchangeable with conventional natural gas and thus can be used in natural gas vehicles. For example, the RNG produced via the processes described herein can be injected into pipeline or used as conventional natural gas. Pipeline-quality gas is a gas product that typically contains of 70-98% by volume methane and varying amounts of other higher alkanes, such as ethane (1.5-9% by volume), propane (0.1-1.5% by volume), butane (0-1% by volume), and pentane (0-1% by volume). Pipeline-quality gas can also include a small percentage of carbon dioxide (0.05-1.0% by volume), nitrogen (0.2%-5.5% by volume), and/or trace amounts of odorizing agents, such as tetrahydrothiophene and mercaptan.

RNG is essentially biogas (the gaseous product of the decomposition of organic matter) that has been processed to purity standards. Like conventional natural gas with similar heating value, RNG can be used as a transportation fuel in the form of compressed natural gas (CNG) or liquefied natural gas (LNG). RNG qualifies as an advanced biofuel under the Renewable Fuel Standard.

RNG is produced from various biomass sources through a biochemical process, such as anaerobic digestion, or through thermochemical means, such as gasification. With appropriate cleanup, biogas or RNG can be used for transportation and other applications. This cleanup process is called conditioning or upgrading, and involves the removal of water, carbon dioxide, hydrogen sulfide, and other trace elements. The resulting RNG, or biomethane, has a higher content of methane than raw biogas, which makes it comparable to conventional natural gas and thus a suitable energy source in applications that require pipeline-quality gas.

Typical sources of biogas and RNG include landfills, biogas from livestock operations, biogas from wastewater plants and other sources of biogas including organic waste from industrial, institutional, and commercial entities, such as food manufacturing and wholesalers, supermarkets, restaurants, hospitals, and educational facilities. But biogas from all such sources have their unique challenges for collection and purification before this can be used as RNG for transportation applications.

To address the current environmental needs of energy production, provided herein are systems and methods for producing ultra-pure green hydrogen and clean renewable natural gas. The processes discussed herein allow for simultaneous production of green hydrogen and clean renewable gas from the same plant in economically viable large quantities using biomass waste. Carbon associated with this biomass is already in circulation and hence is renewable. As such, the green hydrogen and RNG produced by the process discussed herein are carbon neutral. The systems and methods may also produce carbon negative green hydrogen and RNG by sequestration of large quantities of carbon dioxide separated during the biomass gasification process. Accordingly, the green hydrogen and RNG produced by the systems and methods discussed herein can be negative carbon emission fuels for the transportation sector with very low (negative) carbon intensity.

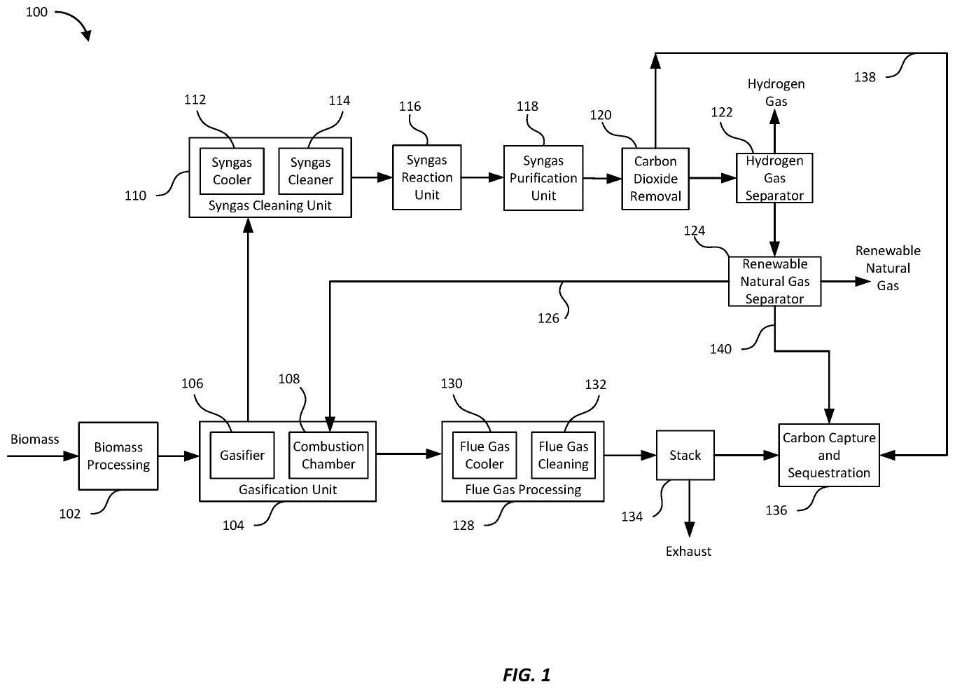

The systems and methods described herein for producing hydrogen and carbon-negative renewable natural gas from biomass may include a gasification step, a reaction step, and a separation step. For ease of discussion, each of these steps will be discussed in turn and further detail is provided below with reference to FIG. 1.

Gasification Step

The gasification step involves gasifying biomass in a gasification unit to form syngas. Gasification is a thermochemical process by which biomass is converted into syngas. Conversion of the biomass into syngas occurs in several phases; namely, a heating phase and a reaction phase. During the heating phase, the biomass is dried to reduce the moisture content within the biomass. For example, water contained in the biomass may be evaporated off by elevating the temperature.

During the reaction phase, several chemical reactions occur. The gasification process generally consists of the following three reactions:

C+H₂O→CO+H₂

C+CO₂→2CO

C+2H₂→CH₄

Depending on the equilibrium, kinetics, and retention time of these reactions, the resulting syngas consists of the following main components: hydrogen gas (H₂), methane (CH₄), ethylene (C₂H₄), carbon monoxide (CO), carbon dioxide (CO₂), and water (H₂O). In some embodiments, the syngas may also include ethane (C₂H₆).

Unlike conventional syngas production, the reaction kinetics of the present process produces ethylene in addition to the main syngas components. In some embodiments, the gasification step may produce from 1-5% by volume of ethylene in the syngas.

In some embodiments, steam may be added during the gasification step as a gasifying agent. Adding steam as a gasifying agent during the gasification process can reduce tar production during the gasification step. The syngas produced by the gasification step discussed herein is nearly nitrogen free and has a high burning efficiency.

Reaction Step

To enrich the hydrogen concentration in the syngas, the process herein includes a carbon monoxide-shift (“CO-shift”) reactor. The CO-shift reactor, also known as a water-gas shift reactor, can increase the overall hydrogen gas yield of the process. For example, the CO-shift reactor can increase the hydrogen gas yield of the process by approximately 30 percent.

The CO-shift reactor reacts the carbon monoxide present in the syngas with water in the presence of a catalyst to form additional carbon dioxide and hydrogen. The following is the main reaction that occurs during the CO-shift reaction.

CO+H₂O↔CO₂+H₂

Addition of water to the syngas before or during the reaction step, drives the above reaction to produce carbon dioxide and hydrogen gas from the carbon monoxide present in the syngas. In some embodiments, steam is added to the syngas stream before the syngas stream enters the CO-shift reactor.

Additionally, the CO-shift reactor may hydrogenate the ethylene present in the syngas to form ethane. Hydrogenation of the ethylene to ethane may be done in the presence of a catalyst. In some embodiments, approximately 75% of the ethylene present in the syngas is converted to ethane. For example, from 40-95% by volume, from 50-95% by volume, from 60-95% by volume, or up to 99.9% by volume of the ethylene may be converted to ethane.

Separation Step

After the hydrogen gas content in the syngas is enriched to form a hydrogen enriched syngas, the hydrogen enriched syngas is separated to form a hydrogen stream and a natural gas stream. The hydrogen stream may be a stream that comprises primarily hydrogen gas or has a hydrogen gas concentration that is greater than the hydrogen enriched syngas stream. For example, the hydrogen gas concentration in the hydrogen stream may range from 90% to approximately 100% by volume.

The natural gas stream may be a stream that contains primarily methane and ethane. In some embodiments, the natural gas stream has a methane concentration that is greater than the hydrogen enriched syngas stream. In other embodiments, the natural gas stream may have an ethane concentration that is greater than the hydrogen enriched syngas stream. In example embodiments, the methane concentration in the natural gas stream may range from 70% to 85% by volume and the ethane concentration in the natural gas stream may range from 20% to 30% by volume. In some embodiments, the natural gas stream may have an ethylene concentration in a range from 0.1% to 1% by volume.

Turning now to FIG. 1, FIG. 1 shows a system block diagram of a plant process 100 for simultaneously producing green hydrogen and renewable natural gas, according to an embodiment herein. Process 100 includes various processing steps. It is understood that one or more of the following steps may be eliminated from the plant process 100 and that the plant process 100 may not be limited to the illustrated and described arrangement. For example, one or more of the following steps may occur at a sequence different than those provided herein. FIG. 1 is merely an illustrative example of a plant process 100 for producing green hydrogen and renewable natural gas.

As illustrated, the plant process 100 includes a biomass processing step 102. The biomass processing step 102 may include a biomass supply and treatment system. The biomass supply and treatment system may include a biomass loading area into which biomass is delivered from external sources. The biomass supply and treatment system may also include one or more chippers and a biomass dryer. Biomass may be transported from the loading area into the chippers, where the biomass is chipped into smaller pieces. The smaller biomass pieces may then be transported into the biomass dryer where the biomass is dried to remove excess water content. Biomass that is dried is sometimes referred to as treated biomass. Treated biomass may have a water content ranging from 15-30% moisture by weight.

The biomass processing step 102 may also include biomass dosing and feeding systems. Biomass dosing and feeding systems are designed to feed treated biomass continuously in the system. For example, treated biomass may be fed into a gasifier unit 106 that is part of the gasification step 104, which is discussed in greater detail below. Exemplary biomass dosing and feeding systems may include lock bins with extraction screws, dosing bins with dosing screws, and feeding screws. The lock bins with extraction screws may extract biomass from the biomass supply and treatment system and feed the biomass to one or more dosing bins. In the dosing bins, the biomass is weighed or otherwise dosed into a desired amount. Once the desired amount of biomass is dosed, the dosed biomass is moved by the feeding screws into the gasification step 104.

Gas produced from the biomass can be hazardous. As such, the biomass dosing and feeding systems provided herein may include equipment and instruments to prevent gas leaking from downstream over pressed areas into the atmosphere or back to upstream biomass transport systems. For example, each lock bin may have a gas-tight gate valve at the inlet and the outlet, which alternatively operate during movement of the biomass.

To allow for continuous feeding of biomass into the gasification step 104, the biomass dosing and feeding systems may include multiple lock bins. While a first lock bin is in refilling mode, delivering biomass from the biomass supply and treatment system, a second lock bin may be in a feeding mode, feeding biomass into the biomass dosing bins. Once the first lock bin is filled, the second lock bin is emptied and the operation mode switches. Meanwhile, biomass accumulated in the dosing bins is continuously fed into the gasification step 104 via the feeding screws.

At the gasification step 104, the treated biomass is gasified to produce syngas, as described above. The gasification step 104 may include a gasifier 106 and a combustion chamber 108. The gasifier 106 may be a bubbling fluidized bed system. For example, the gasifier 106 may be fluidized by superheated steam injected via a nozzle system at the gasifier's bottom. Biomass may be provided by the biomass dosing and feeding system and once inside the gasifier 106, immediately mixes with the hot bubbling bed material.

The operating conditions and parameters for the gasification step 104 may vary depending on the biomass. For example, the composition of the biomass may vary the operating conditions of the gasification step 104. In some embodiments, the gasification reactions, as provided above, may occur at a temperature ranging from 600° C. to 1200° C. For example, the gasification reaction may occur at a temperature ranging from 650° C. to 1150° C., 700° C. to 1000° C., 750° C. to 950° C., or from 800° C. to 900° C.

Heat is transferred from the bed material to the biomass, thereby cooling the bed material. Produced syngas rises up and leave the gasifier at its top, whereas the bubbling bed material remains in the gasification unit 104. Nonvolatile components of the biomass, such as charcoal, settle on the gasifier 106 floor together with the “cooled” bed material. The nonvolatile components of biomass and the “cooled” bed material may be moved from the gasifier 106 to the combustion chamber 108.

The combustion chamber 108 may be an expanding fluidized bed. The nonvolatile components of biomass may be incinerated, thereby reheating the “cooled” bed material in the combustion chamber 108. The bed material may be circulating bed material, such as for example, olivine sand, and act as a heat carrier to utilize the exothermic reaction of burning the nonvolatile components of biomass in the combustion chamber 108. The reheated bed material may then be circulated back into the gasifier 106 where it provides the energy for the endothermic gasification reactions necessary to produce syngas.

During startup of the processing plant 100, a minor flow of syngas may be introduced into the combustion chamber 108. For example, a minor flow of syngas ranging from 0.5%-1.5% by volume of the syngas can be added to the combustion chamber 108 during startup to heat the combustion chamber 108 to the operating temperature. In some embodiments, additional heating fuels may be used during start up, such as injection natural gas.

The operating temperature in the combustion chamber 108, specifically within the combustion zone of the combustion chamber 108, may range from 500° C. to 1500° C., from 600° C. to 1400° C., from 700° C. to 1300° C., from 800° C. to 1200° C., from 900° C. to 1100° C., or from 950° C. to 1000° C. In some embodiments, incinerating the nonvolatile components of biomass may not achieve a temperature necessary to reheat the bed material. Thus, a certain amount of produced syngas may be recycled, in some embodiments on a continuous basis, to the combustion chamber 108.

As the bed material is reheated, it rises in the combustion chamber 108 along with a flue gas. The reheated bed material may be separated from the flue gas by a cyclone. The cyclone may be installed at the top of the combustion chamber 108. Due to the high temperatures of the gasification unit 104, components of the gasifier 106 and the combustion chamber 108 may be designed as welded steel shell construction thermally covered by an inner refractory lining.

To facilitate combustion in the combustion chamber 108, combustion air may be introduced. For example, ambient air may be provided into the combustion chamber 108 by a combustion air fan. In some embodiments, the combustion air may be heated to a temperature ranging from 200° C. to 700° C., from 250° C. to 650° C., from 300° C. to 600° C., from 350° C. to 550° C., or from 400° C. to 500° C. via air pre-heaters. The hot combustion air may then be utilized in the combustion chamber 108 and in a post-combustion chamber, if present. In some embodiments, a certain amount of pre-compressed combustion air may be further compressed by a bottom air fan and injected into the bottom of the combustion chamber 108 to create the expanding fluidized bed system.

The plant process 100 may include a syngas fan, such as a centrifugal fan, that sucks the syngas from the gasification unit 104 through the downstream syngas steps (e.g., 110, 116, 118, 120, etc.).

As noted above, steam may be added as a fluidizing agent and/or a gasifying agent during the gasification process. For example, super-heated steam may be injected into the gasifier 106 via nozzles at the bottom of the gasifier 106. Depending on the size of the gasifier 106 and amount of biomass being gasified, the amount of steam added as a fluidizing and/or gasifying agent may range from 10 to 15% by weight of input biomass.

The syngas produced in the gasifier 106 may exit the gasifier 106 at its top. The syngas may be fed from the gasification unit 104 to a syngas cleaning unit 110. Due to the exit temperature of the syngas, the syngas cleaning unit 110 may include a syngas cooler 112. The exit temperature of the syngas may range from 600° C. to 1050° C., from 650° C. to 1000° C., from 700° C. to 950° C., from 750° C. to 900° C., or from 800° C. to 850° C. The syngas cooler 112 may cool the entering syngas to a temperature ranging from 50° C. to 400° C., from 50° C. to 350° C., from 50° C. to 300° C., from 100° C. to 250° C., or from 150° C. to 200° C.

In some embodiments, the heat from the syngas may be transferred to a high-temperature cooling system. For example, the syngas cooler may be a tube/shell heat exchanger. In such an example, the syngas may flow vertically through straight pipes from the top of the heat exchanger to the bottom. The pipes of the heat exchanger may be cooled on a surface external to the syngas by a cross-flowing coolant. To avoid fouling caused by tars and dust, the velocity of the syngas through the pipes may be high. Because high velocities of solid-containing gas streams raise the risk of abrasion, the pipes may be covered by special materials, and specific design measures may be used.

The syngas cleaning unit 110 may also include a syngas cleaner 114. Due to type of the biomass, the heating media and the thermal and mechanical stress (e.g., abrasion) caused by the turbulences in the bubble bed of the gasifier 106, there may be particulate dust in the syngas. Dust can be problematic in downstream units, thus, the syngas is cleaned to remove particulate dust. Solid particles, such as charcoal dust, may be separated from the syngas via the syngas cleaner 114.

In some embodiments, the syngas cleaner 114 may be a syngas filter such as a fabric baghouse filter unit. Raw syngas from the gasification unit 104, may be fed, after being cooled, into the filter unit. The dust is then separated by the filter bags, leaving the clean syngas to exit the top of the filter housing. Filter bag cleaning is done by a differential pressure-controlled pulse-jet cleaning system. To not impact the quality of the syngas, nitrogen can be used as a cleaning medium. The separated dust, also known as quick coke, has a high calorific value. Thus, it can be utilized in the combustion chamber 108.

After the syngas is cooled and cleaned, the hydrogen concentration of the syngas may be enriched. As discussed above, a reaction step may be performed to enrich the hydrogen concentration in the syngas. The reaction step may include a syngas reaction unit 116. For example, the syngas reaction unit 116 may include a CO-shift reactor or water-gas shift reactor.

The CO-shift reactor may include two vessel reactors containing a fixed-bed catalyst. In some embodiments, the CO-shift reactor may include more than one catalyst. Each of the two vessel reactors may contain the same catalyst, different catalysts, or some combination of similar and different catalysts. Example catalysts include high temperature shift catalysts, such as, e.g., ShiftMax 120 from Clariant.

A heat exchanger may be positioned between the two vessel reactors. Conversion of carbon monoxide to carbon dioxide is more efficient at lower temperatures. For example, conversion of carbon monoxide to carbon dioxide may be efficient at a temperature ranging from 50° C. to 450° C., 100° C. to 400° C., 150° C. to 350° C., or from 200° C. to 300° C. The CO-shift reaction, however, is an exothermic reaction meaning the reaction adds heat to the system. For example, the syngas leaving a vessel reactor may have a temperature ranging from 200° C. to 550° C., 250° C. to 500° C., 300° C. to 450° C., or from 350° C. to 400° C. The heat exchangers may be operated at a pressure near atmospheric pressure. For example, the heat exchangers may be operated at a pressure ranging from 0.5 bar(g) to 2 bar(g), from 0.75 bar(g) to 1.5 bar(g), or from 1.0 bar(g) to 1.2 bar(g). Thus, the syngas is cooled between the first vessel reactor and the second vessel reactor.

The syngas exiting the CO-shift reactor is hydrogen enriched syngas. For example, the syngas exiting the CO-shift reactor may have a hydrogen concentration of 20-30% by volume. The syngas leaving the CO-shift reactor may include an increased concentration of ethane over the syngas entering the CO-shift reactor. As noted above, ethylene in the syngas may be converted to ethane via the reaction step.

The hydrogen enriched syngas may be further cooled after exiting the CO-shift reactor. For example, the hydrogen enriched syngas may be cooled by a syngas cooler, such as a common bundle heat exchanger. Heat exchanged during the cooling of the hydrogen enriched syngas may be used in downstream units. For example, thermal energy removed from the hydrogen enriched syngas may be used to adjust the inlet temperature for a downstream scrubber unit.

In some embodiments, steam may be added before or during the reaction step. For example, steam may be injected into the syngas stream prior to the syngas feeding into the CO-shift reactor. In some embodiments, steam may be added to the syngas stream between the two vessel reactors. Depending on the size of the CO-shift reactor and amount of syngas being enhanced, the amount of steam added to maintain a steam to carbon ratio (molar ratio) in the range of 1.4 to 1.8.

The hydrogen enriched syngas may require purification to remove impurities, such as tar produced during gasification of the biomass. As such, the process 100 may include a syngas purification unit 118. The syngas purification unit 118 may include a tar separation unit. For example, the syngas purification unit 118 may include a double-stage syngas scrubber unit. In such an example, the double-stage syngas scrubber unit may include a tar-removing scrubber column (“tar scrubber column”). The cooled hydrogen enriched syngas may leave the syngas cooler and enter the tar scrubber column at the bottom. The hydrogen enriched syngas may flow bottom-up through internal packings in the tar scrubber column. Biodiesel may be used as a tar scrubbing agent. The biodiesel may be circulated within the column and flow counter to the upstreaming hydrogen enriched syngas. Packing within the tar scrubber column provides for distribution and contact between the gas and liquid phases.

As part of the syngas purification process, water and tars may be condensed out of the hydrogen enriched syngas. For example, the biodiesel may be cooled prior to injection into the tar scrubber column to assist with water extraction from the hydrogen enriched syngas because cooling leads to condensation of water. A direct heat exchanger may also be used to cool the hydrogen enriched syngas to a temperature ranging from 10° C. to 35° C., 15° C. to 30° C., or from 20° C. to 25° C. Low volatile components like organic sulfur components (e.g., thiophene, mercaptan) and aromatics (e.g., benzene, toluene, naphthalene) may also be removed.

After leaving the tar scrubber column, the biodiesel contaminated with tars and the low volatile components may be condensed into a sediment tank where the oily phase and the condensate phases separate out. The biodiesel accumulates on top of the condensate, passes a weir, and is recirculated to the scrubber column after cooling. The condensate is drained from the bottom of the sedimentation tank and pumped to a condensate evaporator. A small amount of biodiesel and condensate is pumped to the combustion chamber 108 to avoid excessive tar concentrations in the scrubbing liquid.

The syngas purification unit 118 may also include an ammonia removal unit. After tar is removed from the hydrogen enriched syngas, ammonia is removed by a second scrubber, an ammonia scrubber column. The hydrogen enriched syngas leaving the tar scrubber column enters the ammonia scrubber column. The hydrogen enriched syngas flows bottom-up through the internal distribution packings of the ammonia scrubber column. Water is used as the scrubbing agent and is circulated in a counterblow manner to the upstreaming hydrogen enriched syngas.

Water and ammonia may be condensed out of the hydrogen enriched syngas. For example, the water may be cooled prior to injection into the ammonia scrubber column to assist with water extraction from the hydrogen enriched syngas. A direct heat exchange may also be used to cool the hydrogen enriched syngas to a temperature ranging from 5° C. to 35° C., 10° C. to 35° C., 15° C. to 30° C., or from 20° C. to 25° C. upon leaving the ammonia scrubber. In some embodiments, a pH-valve controlled injection of diluted sulfuric acid can be implemented to further improve ammonia separation.

After leaving the ammonia scrubber column, the cleaned hydrogen enriched syngas (“clean syngas”) may be treated in further processing steps. As shown by FIG. 1, plant process 100 may include a carbon dioxide removal step 120. Before the hydrogen gas can be separated from the clean syngas, carbon dioxide may be removed. The carbon dioxide removal step 120 may include a carbon dioxide scrubber column.

The clean syngas may be compressed and fed into the carbon dioxide scrubber column. The clean syngas may enter the bottom of the carbon dioxide scrubber column and leave out the top. A solvent may counter-flow the rising clean syngas. In some embodiments, the solvent may include methyl diethanolamine (MDEA). Packing within the carbon dioxide scrubber column can distribute and provide contact between the gas and liquid phases.

Due to the high operating pressure of the carbon dioxide scrubber column, the carbon dioxide is dissolved in the solvent. The carbon dioxide scrubber column may have an operating pressure ranging from 15 bar(g) to 30 bar(g), from 20 bar(g) to 30 bar(g), or from 25 bar(g) to 30 bar(g). The carbon dioxide scrubber column may be operated around ambient temperature. For example, the operating temperature of the carbon dioxide scrubber column may range from 15-50° C., from 15-30° C., or from 15-25° C. In addition to the carbon dioxide, the solvent also dissolves some or all of the hydrogen sulfide present in the clean syngas. The solvent, together with the carbon dioxide and hydrogen sulfide may be drained from the carbon dioxide scrubber column into a flash tank, where it is depressurized to atmospheric pressure. At that point, most of the dissolved carbon dioxide and hydrogen sulfide is released (e.g., vaporizes).

The carbon dioxide scrubber column removes approximately 95 to 99.9% of the carbon dioxide by volume from the feeding clean syngas. For example, the carbon dioxide scrubber column may remove from 95% to 99.9%, 50% to 99.9%, from 50% to 85%, from 60% to 80%, or from 65% to 75% of the carbon dioxide by volume from the feeding clean syngas. The resulting clean syngas exiting the carbon dioxide scrubber column may have a carbon dioxide concentration of 0.05% to 0.5% by volume.

The amine may be recirculated back into the carbon dioxide scrubber column. Amine may be collected from the bottom of the column and fed to an amine regenerator where it is heated so that the dissolved carbon dioxide and hydrogen sulfide is released. A carbon dioxide scrubber feed pump then reinjects the regenerated amine back into the carbon dioxide scrubber column.

The plant process 100 may include a separation step, as discussed above. In the embodiment illustrated by FIG. 1, the separation step may include a hydrogen gas separator step 122 and a renewable natural gas separator step 124.

The clean syngas may be fed to the hydrogen gas separator step 122 at a temperature ranging from 25° C. to 60° C., from 30° C. to 55° C., from 35° C. to 50° C., from 40° C. to 45° C., or from 45° C. to 50° C. 45° C. and a pressure ranging from 18.5 bar(g) to 21.0 bar(g), from 19.0 bar(g) to 20.5 bar(g), or from 19.5 bar(g) to 20.0 bar(g). The hydrogen gas separator step 122 may separate hydrogen gas from the clean syngas. To separate hydrogen gas (“hydrogen’) from the clean syngas, the hydrogen gas separator step 122 may include a pressure swing adsorption unit. The pressure swing adsorption unit may include two vessels, filled with adsorbent material, such as molecular sieves.

The pressure swing adsorption unit may operate the two vessels in different modes. For example, while one vessel is in adsorption mode, the other vessel is in a desorption mode. During the adsorption mode, the clean syngas may enter the vessel via the bottom. The vessel in adsorption mode may be run at a high pressure. For example, the vessel in adsorption mode may operate at a pressure ranging from 15 to 30 bars. In contrast, the vessel in desorption mode may operate at a maximum pressure of 1 bar-g, 0.5 bar-g, or 0.1 bar-g, depending on process conditions. When the vessel switches to the other mode, the vessel changes operating to operate at the pressure corresponding to the operational mode. Due to high pressure, all components except the hydrogen in the clean syngas may be absorbed by the molecular sieve of the vessel. The hydrogen passes through the molecular sieves and leaves the absorber at its top.

During the desorption mode, the vessel is depressurized. By depressurization, the components absorbed by the molecular sieves are released. The released components of the cleaned syngas exit the hydrogen gas separator step 122 as a tail gas. During normal operation, a minor part of the cleaned syngas is used as a thermal resource to heat the gasification unit 104.

The pressure swing adsorption unit may have a longer residence time. For example, the residence time for clean syngas in the pressure swing adsorption unit may range from 5 to 60 minutes, from 5 to 30 minutes, or from 10 to 15 minutes.

The tail gas from the hydrogen gas separator step 122 may be fed to the renewable natural gas separator 124. In some embodiments, the renewable natural gas separator 124 may include a cryogenic separator. The tail gas may have a high concentration of methane and ethane. For example, the tail gas may have a methane concentration in the range from 70-80 percent by volume and ethane concentration of 20-30% by volume. In the cryogenic separator, the tail gas may be cooled to a temperature ranging from −140° C. to −240° C., −160° C. to −220° C., or from −180° C. to −200° C. At this temperature, methane and other hydrocarbons may condense out of the tail gas and separated as a natural gas stream. The natural gas stream can be used as renewable natural gas or blended with pipeline natural gas for transmission and distribution due to its quality. For example, the natural gas stream may contain high quantities of methane, ethane, and propane, with minimal contaminants.

In some embodiments, the cryogenic separator may be operated under pressure. For example, the cryogenic separator may have an operating pressure of 0.2 bar-g to 35.0 bar-g, from 1.0 bar-g to 30.0 bar-g, or from 5.0 bar-g to 25 bar-g. The cryogenic separator may have a moderate residence time. For example, the residence time for tail gas in the cryogenic separator may range from 30 seconds to 30 minutes, from 2 to 20 minutes, from 2 to 15 minutes, from 3 to 10 minutes, or from 3 to 5 minutes.

The remaining components of the tail gas may be fed from the renewable natural gas separator 124 via line 126 back to the gasification unit 104. For example, the remaining components of the tail gas may be fed to the combustion chamber 108. In some embodiments, the tail gas may be used for heating or combustion purposes in the combustion chamber 108.

The flue gas leaving the combustion chamber 108 may be fed to a flue gas processing step 128. The flue gas processing step 128 may include a flue gas cooler 130 and a flue gas cleaner 132.

The flue gas cooler 130 may cool flue gas as it leaves the combustion chamber 108. The flue gas cooler 130 may cool the flue gas before it enters the flue gas cleaner 132. In some embodiments, the flue gas cooler 130 may include a multi-stage cooling system. For example, the flue gas may initially cooled in a first cooling stage involving a radiation cooler with water-cooled walls (high-temperature cooling system), followed by a second cool stage involving a low-temperature cooling system. In such embodiments, the flue gas stream may flow top-down through the multi-stage cooling system and leave the flue gas cooler 130 at a temperature ranging from 450° C. to 800° C., 500° C. to 750° C., 550° C. to 700° C., or from 600° C. to 650° C.

The flue gas cleaner 132 may include a flue gas filter and/or a DeNOₓ unit. The flue gas filter may first remove any particulate or ash that is present in the flue gas before feeding the flue gas to the DeNOₓ unit. The DeNOₓ unit may be a selective catalytic reduction (SCR) unit for reducing environmental emissions, such as NOR. In some embodiments, ammonia may be injected into the flue gas stream to facilitate a chemical reaction to reduce nitrogen oxide (NO) and nitrogen dioxide (NO₂) concentrations in the flue gas.

Following the flue gas processing 128, a portion or all of the flue gas may be released into the environment via stack 134. The remaining portion of the flue gas may be fed to a carbon capture and sequestration (CCS) system 136. The CCS system 136 may capture waste carbon dioxide produced by the plant process 100. CCS system 136 may capture waste carbon dioxide, transport it to a storage site, and deposit the waste carbon dioxide where it cannot enter the atmosphere. For example, the waste carbon dioxide may be stored in an underground geological formation. The CCS system 136 may capture waste carbon dioxide using a variety of technologies, including absorption, adsorption, chemical looping, membrane gas separation, or gas hydrate technologies. Integration of the CCS system 136 into the plant process 100 results in net negative carbon emissions.

Additional streams from process 100 may also be fed to the CCS system 136. For example, a carbon dioxide waste stream 138 from the carbon dioxide scrubber column may be fed to the CCS system 136. In some embodiments, the a waste stream 140 from the cryogenic separator may be fed to the CCS system 136. The waste stream 140 may contain carbon dioxide separated from the tail gas stream during the cryogenic separation. The CCS system 136 may capture the waste carbon dioxide from these additional streams.

Although not depicted, the plant process 100 may include a flare. In the event of a malfunction or shutdown of any subsystems of the plant process 100, the gasifier 106 may continue to produce syngas, even when the fuel feeding is stopped. Subsystems, such as the renewable natural gas separator 124, may also continue to produce gases that cannot be utilized or processed due to the malfunction or shutdown. In such events, various gas streams may be sent to the flare for combustion and to reduce the plant process pressure. As releasing burnable gases into the atmosphere is not allowed, the flare allows for combustion of the purged gases into the environment. During standard operation, no syngas or tail gas is directed to the flare. Instead, the flare is permanently on standby until a malfunction or shutdown event occurs.

All patents, patent publications, patent applications, journal articles, books, technical references, and the like discussed in the instant disclosure are incorporated herein by reference in their entirety for all purposes.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially” of and “consisting of” those certain elements. As used herein, “and/or” refers to and encompasses any and all possible combinations of one or more of the associated listed items, as well as the lack of combinations where interpreted in the alternative (“or”).

It is to be understood that the FIGURE and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure-.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

Different arrangements of the components depicted in the drawings or described above, as well as components and steps not shown or described are possible. Similarly, some features and sub-combinations are useful and may be employed without reference to other features and sub-combinations. Embodiments of the disclosure have been described for illustrative and not restrictive purposes, and alternative embodiments will become apparent to readers of this patent. Accordingly, the present disclosure is not limited to the embodiments described above or depicted in the drawings, and various embodiments and modifications can be made without departing from the scope of the claims below.

That which is claimed is:

1. A method for producing carbon-negative hydrogen and renewable natural gas from biomass, the method comprising:
gasifying biomass in a gasification unit to form a first stream comprising syngas, wherein the syngas comprises methane, hydrogen, carbon dioxide, carbon monoxide, ethylene, and water;
reacting the carbon monoxide with water in the presence of a catalyst to form a second stream, wherein the second stream comprises a greater hydrogen concentration than the first stream; and
separating at least a portion of the second stream to form a hydrogen stream and a natural gas stream, wherein the hydrogen stream has a greater concentration of hydrogen than the second stream, and wherein the natural gas stream has a greater concentration of methane than the second stream.

2. The method of claim 1, wherein the separating step comprises:
separating at least a portion of the second stream into the hydrogen stream and a tail gas stream; and
separating the natural gas stream from the tail gas stream.

3. The method of claim 2, wherein the separating step comprises a pressure swing adsorption process.

4. The method of claim 2, wherein the separating step comprises a cryogenic separation step.

5. The method of claim 1, wherein the method further comprises adding steam to the first stream prior to the reacting step.

6. The method of claim 1, wherein the method further comprises:
capturing waste carbon dioxide; and
sequestering the waste carbon dioxide such that the production of the carbon-negative hydrogen and the renewable natural gas from biomass is a net-negative carbon emission process.

7. The method of claim 1, wherein the natural gas stream comprises a methane concentration of 70-80% by volume.

8. The method of claim 1, wherein the natural gas stream is a pipeline-quality gas that is interchangeable or compatible to be blended with conventional natural gas.

9. The method of claim 1, wherein the reacting step further comprises: hydrogenating the ethylene in the presence of the catalyst to form ethane, wherein the second stream comprises a greater ethane concentration than the first stream.

10. The method of claim 1, the method further comprising removing at least one of tar or ammonia from the first stream prior to the reacting step.

11. A system for producing carbon-negative hydrogen and renewable natural gas from biomass, the system comprising:
a gasification unit, wherein the gasification unit gasifies biomass to form a first stream comprising syngas and a flue gas stream, wherein the syngas comprises methane, hydrogen, carbon dioxide, carbon monoxide, ethylene and water;
a syngas reaction unit, wherein the syngas reaction unit reacts the carbon monoxide with water in the presence of a catalyst to form a second stream, wherein the second stream has a greater hydrogen concentration than the first stream;
a hydrogen extraction unit, wherein the hydrogen extraction unit separates at least a portion of the second stream to form a hydrogen stream and a tail gas stream, wherein the hydrogen stream has a greater concentration of hydrogen than the second stream; and
a natural gas separation unit, wherein the natural gas separation unit separates the tail gas stream to form a natural gas stream, wherein the natural gas stream comprises a greater concentration of methane than the second stream.

12. The system of claim 11, wherein the gasification unit comprises:
a gasifier and a combustion chamber; and
the gasifier is fluidized by superheated steam generated by combusting a portion of the flue gas stream in the combustion chamber.

13. The system of claim 11, wherein the syngas reaction unit comprises a CO-shift reactor.

14. The system of claim 11, wherein prior to introducing the first stream to the syngas reaction unit, a steam stream is added to the first stream.

15. The system of claim 11, wherein prior to the hydrogen extraction unit the system comprises a carbon-dioxide separation unit, wherein:
the carbon-dioxide separation unit comprises at least a carbon-dioxide scrubber; and
the carbon-dioxide separation unit removes at least a portion of carbon dioxide from the second stream.

16. The system of claim 11, wherein the system further comprises a syngas cleaning unit comprising at least one scrubber, wherein the at least one scrubber removes from the second stream at least one of: tar or ammonia.

17. The system of claim 11, wherein the hydrogen extraction unit comprises a pressure swing adsorption unit.

18. The system of claim 11, wherein the natural gas separation unit comprises a cryogenic separation unit.

19. The system of claim 11, the system further comprising a carbon capture and sequestration unit, wherein the carbon capture and sequestration unit captures carbon dioxide produced by the system and removes the carbon dioxide from the system to result in the system having a net negative carbon emission.

20. The system of claim 11, wherein the system further comprise a flue gas processing unit, wherein the flue gas processing unit receives flue gas from the gasification unit and cleans the flue gas.
